(19) United States
(12) Patent Application Publication
(10) Pub. No.: US 2016/0009828 A1
(43) Pub. Date: Jan. 14, 2016

(54) PREPARATION METHOD FOR HYALURONIC ACID, AND ANTI-ADHESIVE COMPOSITION COMPRISING HYALURONIC ACID PREPARED BY SAME PREPARATION METHOD

(71) Applicant: ILDONG PHARM CO., LTD., Seoul (KR)

(72) Inventors: Dae-Jung KANG, Yongin-si (KR); Tae-Yoon Kim, Hwaseong-si (KR); Jae-Hoon Kang, Seoul (KR)

(21) Appl. No.: 14/859,975

(22) Filed: Sep. 21, 2015

Related U.S. Application Data
(63) Continuation of application No. PCT/KR2014/002364, filed on Mar. 20, 2014.

(30) Foreign Application Priority Data
Mar. 20, 2013 (KR) 10-2013-0029876

(51) Int. Cl. C08B 37/08; C12P 19/26; C12R 1/46; A61K 31/728

(52) U.S. Cl. CPC C08B 37/0072; A61K 31/728; C12P 19/26; C12R 1/46

(57) ABSTRACT

A method for hyaluronic acid having a low degradation rate in a subject body includes culturing Streptococcus dysgalactiae strain 9103 (KCTC11818BP) in a medium including a carbon source and a nitrogen source.

### CROSS-REFERENCE TO RELATED APPLICATIONS

This application is a continuation of International Application PCT/KR2014/002364, filed on Mar. 20, 2014, and claims priority from and the benefit of Korean Patent Application No. 10-2013-0029876 filed on Mar. 20, 2013, each of which is hereby incorporated by reference for all purposes as if fully set forth herein.

### BACKGROUND

1. Field

The present disclosure relates to a preparation method for hyaluronic acid possessing a low degradation rate in a body, and an anti-adhesive composition comprising is hyaluronic acid prepared by said preparation method. More specifically, the present disclosure relates to a preparation method for hyaluronic acid possessing a low degradation rate in the body, comprising a step of culturing Streptococcus dysgalactiae strain ID9103 (KCTC11818BP) in a medium comprising a carbon source and a nitrogen source, and an anti-adhesive composition comprising hyaluronic acid prepared by said preparation method.

2. Discussion of the Background

Hyaluronic acid ((HA), Hyaluronan, (C₁₄H₂₀NNaO₁₁) (n>1000)) is a polymer existing in living organisms, and is a polysaccharide, called glycosaminoglycan. It has a structure which is composed of alternating D-glucuronic acid and N-acetylglucosamine, linked together via alternating β-1,3 and β-1,4 glycosidic bonds. It is a water-soluble material with significantly high viscosity and elasticity, while its molecular weight ranges variably from 1,000 to 10,000,000Da (daltons) with its extensive structure of straight chain.

Hyaluronic acid possesses a high efficacy and effectiveness as a lubricant in a physical friction state due to its high moisturizing effect. Also, it has preferable advantages in various effects and properties such as protection against bacterial invasion, leading to its usefulness for a wide range of indications. In order to develop hyaluronic acid, a biological tissue extraction method or a microorganism culturing method has been used basically. However, since a chicken comb extraction method causes many disadvantages such as virus invasion, impurities, and inflammatory reactions, a microorganism culturing production method has become recently a main one in which a molecular weight and productivity can be controlled, and a high quality of raw materials can be obtained. Especially, specific use of hyaluronic acid tends to be determined in accordance with the range of a molecular weight of hyaluronic acid adjusted and produced by the microorganism culturing method. An ultra-low molecular weight hyaluronic acid of 100,000 Da or less is mainly used for foods or cosmetics, while a low molecular weight hyaluronic acid is with an average molecular weight of 1 million Da is utilized for developing an eye-drops raw material or its derivative. Hyaluronic acid with an average molecular weight of 3 million to 4 million Da is highly valuable when utilized as a raw material for a knee joint injection. In addition, its utilization as an ophthalmic surgery adjuvant has been increasing. As an ultra-high molecular weight material in the body, it has been highlighted for the purpose of a raw material for anti-adhesive agent.

Adhesion may be detected generally during a healing process from inflammation. Formation of adhesion in affected tissues occurs through clustering together or abundantly deposited fibrin when granulation tissue or scar forms. In general, an average of 67-93% among laparotomized patients develops adhesion. While spontaneously dissolving in some cases, adhesion maintains in most cases even after wound healing, causing various complications (See Eur. J. Surg. 1997, Suppl 577, 32-39). In order to prevent the formation of adhesion, wrapping around wound areas following surgery has been used, while anti-adhesion agents have been developed around the world, which block the formation of adhesion with surrounding tissues physically or chemically through their pharmacological functions or the like. Such anti-adhesion agents are produced by utilizing various high molecular materials among which arginic acid, CMC and hyaluronic acid are frequently used. As hyaluronic acid, cross-linked hyaluronic acid is utilized of which molecular weight, viscosity, elasticity and the like are enhanced from a low molecular weight hyaluronic acid. While anti-adhesion agents in the form of thin film product are commercially available, it is difficult to straighten the thin film and divide a single film product for the purpose of using in multiple areas. Hence, anti-adhesion agents in the form of gel have been developed which intend to be injected into surgical areas through a syringe. Due to a gel product's property of flowing down, a high molecular weight material is used for the gel is product. In the case of hyaluronic acid, cross-linked hyaluronic acid is commonly used. Meanwhile, when cross-linked hyaluronic acid is utilized as a raw material for an anti-adhesion agent, what matters most is compounds used for cross-linking which may remain and cause an adverse effect in the body even after the degradation of hyaluronic acid as a biomaterial.

Regarding major patented domestic inventions in relation to an anti-adhesion composition comprising hyaluronic acid, Korean Registered Patent No. 10-1074467 discloses a method for preparing an anti-adhesion composition by mixing L-arginine and hyaluronic acid; Korean Registered Patent No. 10-0374666 and Korean Patent Application Publication No. 10-2011-0114810 disclose a method for preparing hyaluronic acid in the form of gel by using sodium hyaluronate as a salt of hyaluronic acid, respectively. Korean Patent Application Publication No. 10-2009-0012439 discloses the use of cross-linked hyaluronic acid prepared by mixing hyaluronic acid and a cross-linking agent. U.S. Pat. No. 6,630,167 discloses a preparation of an anti-adhesion composition by mixing a solution of hyaluronic acid and a solution of a cross-linking agent. As described above, it has not been found any disclosure in domestic and foreign prior arts in which non-cross linked or non-structurally transformed hyaluronic acid was used singularly. Further, both domestic and foreign prior arts generally expressed negative views on the single use of non-cross linked hyaluronic acid due to its easy degradation and thus short period of stay in the body.

Therefore, it is urgently needed to develop hyaluronic acid which is not cross-linked with long period of stay in the body and an anti-adhesion composition prepared by utilizing said hyaluronic acid.

### SUMMARY

Accordingly, the inventors of the present disclosure have researched on a method of producing hyaluronic acid with long stay in the body and high efficacy for anti-adhesion to find that hyaluronic acid prepared by using Streptococcus dysgalactiae strain ID9103 is effective for inhibiting adhesion due to its low degradation rate in the body, leading to the completion of the present invention.

An object of the present disclosure is to provide a method for preparing hyaluronic acid with low degradation rate in the body, comprising a step of culturing Streptococcus dysgalactiae strain ID9103 (KCTC11818BP) in a medium comprising a carbon source and a nitrogen source.

Another object of the present disclosure is to provide a composition for inhibiting adhesion comprising non-cross linked hyaluronic acid prepared by said method as an active ingredient.

Still another object of the present disclosure is to provide a method for inhibiting adhesion comprising a step of administering non-cross linked hyaluronic acid prepared by said method to a subject in need thereof

Further still another object of the present disclosure is to provide non-cross linked hyaluronic acid prepared by said method and used for inhibiting adhesion.

To achieve the above-mentioned objects, an exemplary embodiment provides a method for preparing hyaluronic acid with a low degradation rate in the body, comprising a step of culturing Streptococcus dysgalactiae strain ID9103 (KCTC11818BP) in a medium comprising a carbon source and a nitrogen source.

To achieve another above-mentioned object, an exemplary embodiment provides a composition for inhibiting adhesion comprising non-cross linked hyaluronic acid prepared by said method as an active ingredient.

To achieve still another above-mentioned object, an exemplary embodiment provides a method for inhibiting adhesion comprising a step of administering non-cross linked hyaluronic acid prepared by said method to a subject in need thereof

To achieve further still another above-mentioned object, an exemplary embodiment provides non-cross linked hyaluronic acid prepared by said method and used for inhibiting adhesion.

### BRIEF DESCRIPTION OF THE DRAWINGS

FIG. 1 is a picture showing the effect of hyaluronic acid in inhibiting adhesion based on its molecular weight in the open-cut abdomen of test animals.

FIG. 2 is a graph comparing the degradation rate of hyaluronic acid over time (Y axis: viscosity (cP); X axis: minutes (min)).

### DETAILED DESCRIPTION OF THE ILLUSTRATED EMBODIMENTS

Hereinafter, exemplary embodiments will be described in detail.

A method for preparing hyaluronic acid with a low degradation rate in the body may include a step of culturing Streptococcus dysgalactiae strain ID9103 (KCTC11818BP) in a medium including a carbon source and a nitrogen source.

Preferably, the preparation method includes following steps:
(a) culturing Streptococcus dysgalactiae strain ID9103 (KCTC11818BP) in a medium including a carbon source and a nitrogen source; and
(b) collecting hyaluronic acid from the resulting culture in the medium of step (a).

The Streptococcus dysgalactiae strain ID9103 (accession number : KCTC-11818BP; U.S Pat. Pub. No. 2014/0206040, which is herein incorporated by reference, specifically with respect to description of Streptococcus dysgalactiae strain ID9103) is a microorganism which was isolated by separating hyaluronic acid-producing strains among microorganisms separated from cow feces, followed by causing mutation in the strains and then selecting a non-hemolytic strain that does not produce hyaluronidase.

Selection of Streptococcus dysgalactiae ID9103

<1-1> Securing of Strain of Genus Streptococcus Producing Hyaluronic Acid

Genus streptococcus producing hyaluronic acid (HA) is well grown in a brain heart infusion medium (Calf brains, infusion 0.77%, beef hearts, infusion 0.98%, proteose peptone 1%, dextrose 0.2%, NaCl 0.5%, Disodium phosphate 0.25%; BD, US), and in single colony separation, it can produce hyaluronic acid, thereby forming more smooth and viscous colonies than general colonies.

About 500 samples collected from 10 stables of the whole country were diluted in such a manner that about 200 colonies can be grown per solid medium, and smeared on 3.7% brain heart infusion solid medium. Then, with the naked eye, colonies that were found to produce a viscous substance were selected.

In order to morphologically observe separated strains, they were smeared on a brain heart infusion solid medium so as to separate colonies, and cultured in a 37° C. culture medium. When the colonies were formed, one drop of sterilized distilled water was dropped on a slide glass. Then, one colony was placed on the tip of a sterilized toothpick and dissolved in distilled water. It was covered with a cover glass, and coccus with chain structure, like genus streptococcus, was selected by enlargedly observing microbial cells through a microscope (x400).

In order to select gram-positive genus streptococcus, a sample on a slide glass was prepared through a gram staining method in the above described method, and was slightly heated on a lamp so as to attach bacteria on the slide glass. About 1 minute later after staining with dye, the dye was washed with slightly flowing water. When the water was dried to some extent, one drop of mineral oil was dropped thereto. The sample was covered with a cover glass, and observed by a microscope. Herein, the stained gram-positive bacteria are colored violet. Through this method, suitable strain candidates were selected.

In order to determine if a viscous substance produced by each strain is HA, HA production was confirmed. In order to confirm the production of HA, a carbazole reaction and a cetyltrimethyl-ammonium bromide (CTAB) reaction are used. The carbazole reaction is a method of measuring the amount of glucuronic acid produced by decomposition of HA by sulfuric acid. After the production of HA by the carbazole reaction was confirmed, the CTAB reaction was carried out so as to confirm HA production. CTAB destroys a mucous membrane and makes it opaque. HA is a viscous substance, and thus forms an insoluble complex and becomes opaque by being destroyed by CTAB. The carbazole reaction has a disadvantage in that since glucuronic acid produced by other sugars is measured, HA in a higher amount may be measured in a culture solution state than in an actual amount. Since CTAB reacts with only HA, it is possible to simply confirm HA production within a short time. Through the carbazole reaction, strains producing polysaccharide including HA were selected, and from among the strains, through the CTAB reaction, a strain producing HA was selected.

The carbazole reaction is a method in which uronic acid can be quantitated. Glucuronic acid, one of materials constituting hyaluronic acid, is colored purple by the reaction, and thus can be quantitated. In the carbazole reaction, 1 ml of a sample was dissolved in 5 ml of is 0.025M (in H₂SO₄) sodium tetraborate decahydrate, sufficiently mixed, and boiled in water for 10 minutes. After being cooled in ice, it was added and mixed with 200 ul of 0.1% (in EtOH) Carbazole, and boiled in water for 10 minutes. At 525 nm, the absorbency was measured.

In the CTAB reaction, 1/10-diluted culture solution was diluted again to half concentration with 0.03% SDS solution. Then, 200 ul of the resultant solution was mixed with 200 ul of acetic acid buffer (sodium acetate 1.55%, acetic acid 0.063%, NaCl 0.88%), and reacted at 37° C. for 30 minutes. 800 ul of CTAB solution was added thereto. At 600 nm, the absorbency was measured.

<1-2> Securing of Non-Hemolytic Mutant Strain with no Hyaluronidase Activity

The hyaluronic acid-producing strain selected in Example <1-1> was shake-cultured in 50 ml of 3.7% brain heart infusion liquid medium for 24 hours at 37° C. A culture solution with OD (600) of 0.3 was treated with N-Methyl-N′-nitro-N-nitrosoguanidine (NTG), followed by stirring at 37° C. for 1 hour so as to determine the condition at a lethal rate of 95%. The culture solution treated with 10 mg/ml of NTG was centrifuged at a rotation speed of 4000 rpm for 10 minutes, and the microbial cells were collected and washed with 50 mM Tris-maleate buffer (pH 8.0) three times. The spores on which mutation was induced were diluted with sterilized saline solution at a concentration of 10²˜10⁴/ml, and were smeared on a brain heart infusion solid medium including 5% sheep blood and cultured at 37° C. Then, non-hemolytic colonies without a clear zone made by destruction of erythrocytes were selected. However, because hemolyticity may occur again, NTG mutation for such non-hemolytic colonies was repeatedly performed. Then, colonies that do not show hemolyticity in subculture were selected.

From among the secured non-hemolytic strains, colonies that do not express is hyaluronidase enzyme were selected by CTAB reaction described in Example 1-1. The secured non-hemolytic strains were cultured for one day in a brain heart infusion solid medium added with 0.1% hyaluronic acid, and 10% CTAB was added to the upper layer. From among colonies with no hyaluronidase activity, Streptococcus dysgalactiae ID9103 having no clear zone around itself was selected.

<1-3> Identification of Selected Strain

In order to identify the strain as genus streptococcus based on biochemical characteristics in Bergey's manual, in an identification experiment, a basic medium including yeast extract and peptone was added with sugar or amino acid required for determining biochemical characteristics, and the color change was changed. The sources added to the medium comprise inulin, lactose, mannitol, raffinose, ribose, salicin, sorbitol, trehalose, arginine, esculin, and hippurate. Herein, bromperesol purple (BCP)was added thereto, and the color change between the strain and a non-inoculated control was observed. BCP is colored violet at neutral pH, yellow at acidic pH, and red at basic pH. Before being inoculated with microbial cells, the medium is neutral, and colored violet.

As a result, the inventive Streptococcus dysgalactiae ID9103 strain showed a highly similar characteristic to dysgalactiae of genus streptococcus based on Bergey's manual. This result is noted in Table 1.

TABLE 1
Biochemical characteristics

| | Inulin | Lactose | Mannitol | Raffinose | Ribose | Trehalose | Arginine | Esculin | Hippurate |
|---|---|---|---|---|---|---|---|---|---|
| Control | − | + | − | − | + | + | − | − | − |
| group | | | | | | | | | |
| Test | − | + | − | − | + | + | + | − | − |
| group | | | | | | | | | |

The Streptococcus dysgalactiae ID9103 strain selected in Example <1-2> was identified using an Api kit. By using an Api 20 strep kit (Biomerieux, France) for identification of streptococcus, the identification was performed following the manufacturer's manual. On a solid medium, the strain was sufficiently dissolved in 2 ml of suspension medium by using a cotton swab so as to prepare inoculation liquid with 4 McFarland turbidity. The strep comprises a total of 20 cupules including VP, HIP, ESC, PYRA, αGAL, βGUR, βGAL, PAL, LAP, ADH, RIB, ARA, MAN, SOR, LAC, TRE, RAF, AMD, and GLYG in order, and they cause different reactions, respectively. To VP to LAP each, the inoculation liquid was filled in an amount of 100 ul, and to others, the inoculation liquid in an amount of 500 ul mixed with 2 ml of GP medium was filled in an amount of 100 ul. ADH and GLYG were added with mineral oil. After culturing for 4 hours, VP was added with one drop of VP1 and VP2 each, HIP was added with two drops of NIN reagent, and PYRA to LAP were added with one drop of ZYM A, and ZYM B reagents each. After 10 minutes, the results were measured, and after 24 hours, the results on other cupules were read again.

As a result, it was identified Streptococcus dysgalactiae ID9103 is dysgalactiae of genus streptococcus.

The Streptococcus dysgalactiae strain ID9103 according to an exemplary embodiment may be cultured by conventional methods for culturing microorganisms of genus streptococcus.

The culture medium may include a carbon source and a nitrogen source. It may further include an amino acid or a metal ion.

There is no limitation to the carbon source, as long as it is a carbon source being used in known microorganism culturing methods. Preferably, it may be selected from the group is consisting of glucose, fructose, maltose, lactose, galactose, glycerol and a mixture thereof. More preferably, it may be maltose.

There is no limitation to the nitrogen source, as long as it is a nitrogen source being used in known microorganism culturing methods. Preferably, it may be selected from the group consisting of yeast extract, casein peptone, casein acid hydrolysate, casein enzymatic hydrolysate, bacto-peptone, casitone, neopeptone and a mixture thereof. More preferably, it may be casein enzymatic hydrolysate.

The casein enzymatic hydrolysate is obtained by enzymatic decomposition of casein. For example, it may be tryptone, tryptone T, tryptone X, BBL biosate peptone, DIFCO casein digest, bacto casitone, BBL trypticase peptone, bacto tryptone, Bitec tryptone, NZ amine A, NZ amine AS, NZ amine EKC, NZ amine L concentration, NZ case, NZ case M, NZ case ME, NZ case plus, NZ case TT, pepticase, tryptone USP, pancreatic digest casein codex, pancreatic digest casein, enzymatic hydrolyzed casein kosher, or tryptone V.

The culture medium may further include an amino acid or a metal ion.

There is no limitation to the kind of the amino acid. Preferably, it may be selected from the group consisting of glutamine, lysine, cysteine, arginine, methionine, aspartic acid, glycine and a mixture thereof. More preferably, it may be arginine.

There is no limitation to the kind of the metal ion. Preferably, it may be selected from the group consisting of sodium, potassium, calcium, magnesium, iron, zinc, manganese and a mixture thereof. More preferably, it may be zinc.

More preferably, the culture medium according to an exemplary embodiment may include casein enzymatic hydrolysate as the nitrogen source, arginine as the amino acid, and zinc as the metal ion. While being cultured in the medium including casein enzymatic hydrolysate, arginine and zinc, the molecular weight of hyaluronic acid prepared by said microorganism according to an exemplary embodiment may be modified to obtain hyaluronic acid with a molecular weight producing the most effective anti-adhesive function.

There is no limitation to the concentration of casein enzymatic hydrolysate, arginine and zinc, respectively. Preferably, the casein enzymatic hydrolysate may be included at a concentration of 0.5% (w/v) to 3% (w/v), arginine at a concentration of 0.01% (w/v) to 0.6% (w/v), and zinc at a concentration of 0.01% (w/v) to 0.1% (w/v).

There is no specific limitation to the culture method according to an exemplary embodiment. For example, batch, fed-batch, or continuous culture methods may be used. According to an exemplary embodiment, a fed-batch culture method may be preferably used. In the fed-batch culturing, a culture medium being supplied to a fed-batch may include a nitrogen source, or both a nitrogen source and a carbon source. More preferably, the nitrogen source is casein enzymatic hydrolysate, and the carbon source is maltose.

The step of collecting hyaluronic acid from the preparation method according to an exemplary embodiment may be conducted through known methods of isolating an active material from microorganism culture. Specifically, such known methods include bacteriostatic processes such as filtration, neutralizing processes, crystallization processes, and processes of removing and isolating impurities such as endotoxins, proteins, nucleic acids and metals (such as chromatography and centrifugation).

Hyaluronic acid prepared by the method according to an exemplary embodiment may preferably include, but not limited thereto, a high molecular weight hyaluronic acid having an average molecular weight of between 3.5 million Da and 10 million Da. More preferably, the high molecular weight hyaluronic acid may be selected from the group consisting of hyaluronic is acids having an average molecular weight range of between 4 million Da with 6 million Da, between 4 million Da and 8 million Da, and between 4 million Da and 10 million Da. Average molecular weight as described herein is weight average molecular weight.

Further, the preparation method according to an exemplary embodiment may lead to the high yield production of high molecular weight hyaluronic acid with specified average molecular weight.

An exemplary embodiments provides a preparation method in which a high molecular weight hyaluronic acid with its molecular weight ranges between 4,320,000 Da and 5,980,000 Da was obtained in a high yield of 8.07 g/L to 9.42 g/L (See Table 1).

The term “low degradation rate” as used herein means that hyaluronic acid is degraded or decomposed at a low rate upon being administered to the body of a subject.

The present disclosure also describes a comparative experiment on the degradation rate of hyaluronic acid prepared by the method according to an exemplary embodiment in the body of a subject. In order to compare the degradation rate of hyaluronic acid in the body, hyaluronic acids having different molecular weights were treated with hyaluronidase which decomposes hyaluronic acid in the body, respectively, followed by measuring a viscosity value (cP) for each hyaluronic acid (3 million Da; 4 million Da; 6 million Da) over time. It was found that hyaluronic acids having the molecular weight of 4 million Da and 6 million Da respectively exhibited lower reduction rate in viscosity over time than one having the molecular weight of 3 million Da. Having low reduction rate in viscosity means that hyaluronic acid degrades or decomposes slowly in the body of a subject and thus is capable of maintaining its viscosity at a certain level. Thus, hyaluronic acid having the molecular weight of 4 million Da or more prepared by the method according to an exemplary embodiment may possess a low is degradation rate in the body of a subject. Preferably, hyaluronic acid having the molecular weight of 4 million Da to 6 million Da may be excellent for inhibiting adhesion.

Hyaluronic acid prepared by the method according to an exemplary embodiment may include hyaluronic acid having an average molecular weight of between 3.5 million Da and 10 million Da, preferably between 4 million Da and 10 million Da, more preferably between 4 million Da with 6 million Da. Hyaluronic acid having an average molecular weight of 3.5 million Da or less possesses high degradation rate in the body and thus lacks its effectiveness in inhibiting adhesion, while hyaluronic acid having an average molecular weight of 10 million Da or more is difficult to prepare.

As described above, hyaluronic acid prepared according to an exemplary embodiment has a low degradation rate in the body of a subject.

Further, an anti-adhesion composition including said hyaluronic acid as an active ingredient has low degradation rate in the body of a subject, resulting in an excellent effect in inhibiting adhesion.

A composition for inhibiting adhesion may include, as an active ingredient, non-cross linked hyaluronic acid prepared by said method according to an exemplary embodiment.

A method for inhibiting adhesion may include a step of administering, to a subject in need thereof, non-cross linked hyaluronic acid prepared by said method according to an exemplary embodiment.

Non-cross linked hyaluronic acid may be prepared by said method according to an exemplary embodiment and used for inhibiting adhesion.

Furthermore, a composition for inhibiting adhesion may include the hyaluronic acid according to an exemplary embodiment as an active ingredient. It may further include is physiological saline solution, distilled water, sodium phosphate buffer solution and so on.

Hyaluronic acid according to an exemplary embodiment has a lower degradation rate in the body of a subject than previously known hyaluronic acids, and thus even in its non-cross linked form possesses lubrication effect for inhibiting adhesion for a sufficient time, resulting in being used as a potent anti-adhesion agent.

The term “non-cross linked hyaluronic acid” as used herein refers to hyaluronic acid prepared by said preparation method according to an exemplary embodiment which is not comprised of cross-linking chemical agents, chemical denaturants or cationic polymers forming a complex with hyaluronic acid.

The term “cross-linking chemical agents” as used herein refers to chemical compounds which react with hyaluronic acid to form a three dimensional network structure, and may be at least one selected from the group consisting of polyvalent epoxy compound such as polyglycidyl ether, divinyl sulfone, formaldehyde, phosphorous oxychloride, a mixture of carbodiimide compound and amino acid ester, and a mixture of carboiimide compound and dihydrazide compound.

The term “chemical denaturants” as used herein refers to chemical compounds which react with carboxyl, hydroxyl or acetamide substituents of hyaluronic acid to form covalent bonds, and may be at least one selected from the group consisting of a mixture of acetic anhydride and concentrated sulfuric acid, a mixture of anhydrous trifluoroacetic acid and organic acid, and alkyl iodine compound.

The term “cationic polymers forming a complex with hyaluronic acid” as used herein refers to polymer compounds which form a complex via ionic bonds between carboxyl substituents of hyaluronic acid and amino or imino substituents of the polymer compounds, and is may be at least one selected from the group consisting of chitosan, polylysine, polyvinylpyridine, polyethyleneimine and polydimethylaminoethylmetacrylate.

The term “subject” as used herein refers to an animal, preferably a mammalian animal including humans, while including cells, tissues or organs originated from an animal. The “subject” may be a patient in need of treatment.

The term “administering” as used herein refers to, but not limited thereto, applying, distributing or attaching to a subject in need thereof a composition for inhibiting adhesion including hyaluronic acid according to an exemplary embodiment as an active ingredient.

The composition for inhibiting adhesion according to an exemplary embodiment may be administered to any body part of a subject, including visceral organs in abdominal and thoracic cavities, paratenons, cranial bones, nerves, bulbus oculi during laparotomy, gynecologic surgery and thoracotomy; tendons and ligaments during orthopedic surgery; and duramater during neurosurgery.

The composition for inhibiting adhesion according to an exemplary embodiment may be used, but not limited thereto, in the form of gel, film or membrane.

A preparation method for hyaluronic acid possessing a low degradation rate in the body, and a composition for inhibiting adhesion including hyaluronic acid prepared by said preparation method are described. Hyaluronic acid prepared by the method has an average molecular weight of between 3.5 million Da and 10 million Da with a potent effect in inhibiting adhesion due to its low degradation rate in the body. Therefore, since the composition for inhibiting adhesion including hyaluronic acid prepared by the method possesses an excellent effect in inhibiting adhesion and utilizes non-cross linked hyaluronic acid, it is very effective in is overcoming drawbacks of conventional hyaluronic acid and conventional anti-adhesion compositions containing cross-linking agents and chemical compounds.

Hereinafter, exemplary embodiments will be described in detail by referring to following examples.

However, the following examples are merely for illustrating exemplary embodiments, and are not intended to limit the scope of the present invention.

### EXAMPLE 1

### Experiment on the Effect of Inhibiting Adhesion Based on the Molecular Weight of Hyaluronic Acid

SD rats (female, SPF, Orient Bio, Inc.) were anesthetized via inhalation and maintained under anesthesia throughout surgery. Their four limbs were tightly fixated on the operating table, followed by the removal of their abdominal hair with the aid of a razor. After disinfectants being applied, their abdomens were incised with operating scissors. Their appendixes were pulled out and rendered to be damaged by using coarse gauzes to the extent that the gauzes were smeared with bloodstains. The abdominal walls where the appendixes were located were also damaged in the same way as the appendixes, causing a condition where adhesion could occur between the appendixes and the internal abdominal walls. Test substances were administered and then the incision areas were sutured. After 2 weeks, laparotomy was performed to check the occurrence of adhesion. Said test substances included hyaluronic acids having an average molecular weight of 3 million Da, 4 million Da and 6 million Da, respectively, dissolved in sodium phosphate buffer solution and maintained under an aseptic condition.

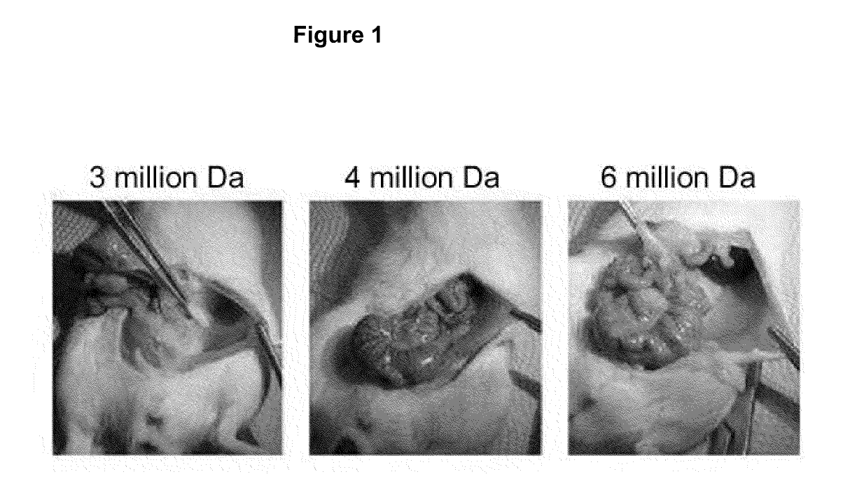

Adhesion between the abdominal organ and the abdominal walls was detected upon using hyaluronic acid of which molecular weight was 3 million Da. On the contrary, upon using hyaluronic acids of which molecular weights were 4 million Da or more, such an adhesion is was not observed (See FIG. 1). Hence, it was confirmed that hyaluronic acid having a molecular weight of between 4 million Da and 6 million Da possesses an anti-adhesion effect.

### EXAMPLE 2

### Basic Culturing Conditions for Producing High Molecular Weight Hyaluronic Acid

4 ml of Streptococcus dysgalactiae strain ID9103 culture solution stored in a −72° C. refrigerator was rapidly thawed, smeared on 5.2% brain heart infusion solid medium, and cultured at 37° C. for 24 hours. The grown colony was cut with an area of 1 cm² and inoculated into 40 ml of 3% Todd-Hewitt broth sterilized liquid medium (heart, infusion 0.31%, neopeptone 2%, dextropse 0.2%, NaCl 0.2%, Disodium phosphate 0.04%, sodium carbonate 0.25%; BD, US).

40 ml of the liquid shake-cultured at 37° C. and 150rpm was used as a primary seed culture solution. In a Logarithmic growth phase following culturing for 6 hours, the primary seed liquid was aseptically inoculated to three 3% Todd-Hewitt broth sterilized liquid media (40 ml, pH 7.8). Under the culturing condition of 37° C. and 150rpm, after aseptic culturing for 20 hours or more, the cultured medium was used as a secondary seed culture solution. Herein, the secondary seed culture solution should be maintained at pH of 6.4±0.2, and have OD (600) of 0.35±0.05. 80 ml of the secondary seed culture solution was inoculated to a main culture medium, followed by culturing for 40 hours or more. The difference among hyaluronic acids in the productivity based on a medium composition was observed. Subsequently, the conditions for increasing the productivity of hyaluronic acid were determined. The culturing processes as described above were performed in the same manner in all Examples.

Tests of determining the main culture medium for optimally producing hyaluronic acid were conducted in a 7.5L fermentation bath under 3.5L culture solution is condition. The medium composition was comprised of glucose 6% (w/v), yeast extract 0.5% (w/v), casein peptone 2% (w/v), glutamine 0.06% (w/v), sodium gluconate 0.1% (w/v), oxalic acid 0.02% (w/v), magnesium sulfate 0.15% (w/v), potassium phosphate dibasic 0.25% (w/v), sodium chloride 0.5%(w/v), sodium acetate 0.5%(w/v), ferric chloride 0.007%(w/v), and ammonium molybdate 0.05% (w/v). The tests were basically performed under the condition of pH 7.0, and 34° C.

According to an exemplary embodiment, the hyaluronic acid concentration in the culture solution was confirmed by both a carbazole method (T. Bitter, Anal. Biochem., 1962, 4, 330-334) and a turbidity analysis (S. Jung-Min, Carbohyd. Polym., 2009, 78, 633-634).

The average molecular weight of hyaluronic acid was obtained by a gel filtration chromatography method (Narlin B. Beaty et al, Anal. Biochem., 1985, 147, 387-395). In the analysis, the column was Toyo Soda TSK gelG6000PWXL, and the moving phase was comprised of 150 mM NaCl, 3 mM Na₂HPO₄ (pH7.0), and 0.02% NaN₂. The detection was performed by a refractive index detector (Shodex; Showa Denko K.K. Japan), and the standard substance was prepared by polyethylene oxide at 2 mg/ml concentration per molecular weight.

Hyaluronic acids prepared in accordance with the above described culturing conditions were detected to have a concentration of 7 g/L and a molecular weight of 3 million Da.

### EXAMPLe 3

### Productivity and Molecular Weight of High Molecular Weight Hyaluronic Acid

It has been known that nitrogen source plays an important role in the metabolism of microorganisms and also affects the production of hyaluronic acid. Hence, the present inventors reasoned that changing the types of amino acids including a class of peptones used as a basic nitrogen source might contribute to the production of hyaluronic acid having a molecular is weight of 4 million Da to 6 million Da as desired.

The present inventors confirmed that the concentration and the molecular weight of hyaluronic acid varied depending on the type of peptones. Among many different kinds of peptones, casein (enzymatic hydrolysate) used as the basic medium source lead to the best outcome, i.e. the production of hyaluronic acid having the concentration of 8 g/L or more and the molecular weight of 4,320,000 Da. Thus, all the experiments in following examples utilized casein (enzymatic hydrolysate).

The test group in which arginine was added instead of glutamine as a basic medium source resulted in an excellent outcome, i.e. hyaluronic acid having the concentration of 8.53 g/L or more and the molecular weight of about 4,830,000 Da was produced.

Carbon source also has been known to play an important role in the growth and metabolism of microorganisms, and has been utilized as a precursor of hyaluronic acids. Hence, the present inventors reasoned that changing the types of carbon source might affect the production of hyaluronic acid having a molecular weight of 4 million Da to 6 million Da as desired.

The test group in which maltose was added instead of glucose as a basic medium source resulted in an excellent outcome, i.e. hyaluronic acid having the concentration of 8.72 g/L or more and the molecular weight of about 5,520,000 Da was produced.

Metal ions have been known to play various roles inside the cells of microorganisms including the expression of DNAs and the activation of enzymes. Hence, under the assumption of their influence in the expression of DNAs and the activation of enzymes involved with the production of hyaluronic acid, various metal ions were tested.

The test group in which zinc was added resulted in an excellent outcome, i.e. is hyaluronic acid having the concentration of 9.42 g/L or more and the molecular weight of about 5,980,000 Da was produced.

TABLE 2
Difference in the concentration and the molecular weight of hyaluronic
acid (HA) according to the type of peptones as a medium source.

| | | Molecular weight |
|---|---|---|
| Type of peptones | Conc. of HA (g/L) | of HA (×1,000) |
| Casein peptone → | 8.07 | 4,321 |
| Casein (enzymatic hydrolysate) | | |
| Glutamine → Arginine | 8.53 | 4,829 |
| glucose → Maltose | 8.72 | 5,523 |
| Zinc (added) | 9.42 | 5,981 |

### EXAMPLE 4

### Comparative Experiments on the Degradation Rate of Hyaluronic Acid in the Body

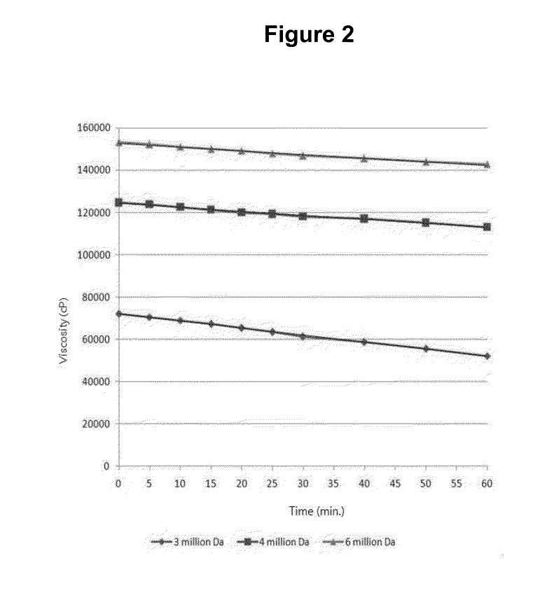

Comparative experiments on the degradation rate of hyaluronic acid having the molecular weight of 3 million Da, 4 million Da and 6 million Da were respectively conducted by using hyaluronidase which degrades or decomposes hyaluronic acid in the body. Comparative experiments on degradation rate were performed to compare the difference in viscosity levels using the measured values (cP) between before and after the treatment with hyaluronidase. The measurement of viscosity was conducted using Brookfield Digital Viscometer LVDV-1+ (Brookfield, USA) with the setting of spindle 31, 0.3 RPM and 25° C. The results are shown in Table 3 and FIG. 2.

TABLE 3
Comparative experiments on the degradation rate of
hyaluronic acid (HA) in the body according to its molecular weight.

| Time after the addition of | 3 million | | 6 million |
|---|---|---|---|
| hyaluronidase (min.) | Da HA | 4 million Da HA | DaHA |
| 0 | 72,100 | 124,800 | 153,200 |
| 5 | 70,700 | 123,900 | 152,300 |
| 10 | 69,100 | 122,700 | 151,200 |
| 15 | 67,300 | 121,400 | 150,100 |
| 20 | 65,400 | 120,100 | 149,300 |
| 25 | 63,600 | 119,300 | 148,100 |
| 30 | 61,200 | 118,200 | 147,000 |
| 40 | 58,700 | 117,100 | 145,700 |
| 50 | 55,600 | 115,300 | 144,100 |
| 60 | 52,300 | 113,200 | 143,000 |
| Viscosity reduction rate | 27.5% | 9.3% | 6.7% |

As shown in Table 3 and FIG. 2, hyaluronic acid of 3 million Da had its viscosity reduction rate of 27.5% which was quite high in comparison with those of hyaluronic acids of 4million Da and 6million Da, respectively. Hyaluronic acids of 4 million Da and 6 million Da, respectively, had about three or four times less viscosity reduction rate than hyaluronic acid of 3 million Da, confirming that hyaluronic acids of 4 million Da and 6 million Da possess lower degradation rate in the body and thus maintain their viscosity levels at a certain level. Thus, it was confirmed that hyaluronic acid having a molecular weight of 4 million Da or more prepared by the preparation method according to an exemplary embodiment possesses a low degradation rate in the body, while, preferably, hyaluronic acid having a molecular weight of between 4 million Da and 6 million Da is excellent in inhibiting adhesion.

What is claimed is:

1. A method for preparing hyaluronic acid with a low degradation rate in the body of a subject, the method comprising a step of culturing Streptococcus dysgalactiae strain ID9103 (KCTC11818BP) in a medium comprising a carbon source and a nitrogen source.

2. The method of claim 1, wherein the carbon source is maltose and the nitrogen source is casein enzymatic hydrolysate.

3. The method of claim 1, wherein the medium further comprises an amino acid or a metal ion.

4. The method of claim 3, wherein the amino acid is arginine and the metal ion is zinc.

5. The method of claim 1, wherein the hyaluronic acid has a molecular weight of between 3.5 million Da and 10 million Da.

6. The method of claim 5, wherein the hyaluronic acid has a molecular weight of between 4 million Da and 6 million Da.

7. A composition for inhibiting adhesion comprising as an active ingredient a non-cross linked hyaluronic acid prepared by culturing Streptococcus dysgalactiae strain ID9103 (KCTC11818BP) in a medium comprising a carbon source and a nitrogen source.

8. The composition of claim 7, wherein the carbon source is maltose and the nitrogen source is casein enzymatic hydrolysate.

9. The composition of claim 7, wherein the medium further comprises an amino acid or a metal ion.

10. The composition of claim 9, wherein the amino acid is arginine and the metal ion is zinc.

11. A method for inhibiting adhesion, the method comprising a step of administering a non-cross linked hyaluronic acid prepared by the method of claim 1 to a subject in need thereof.

12. The method of claim 11, wherein the carbon source is maltose and the nitrogen source is casein enzymatic hydrolysate.

13. The method of claim 11, wherein the medium further comprises an amino acid or a metal ion.

14. The method of claim 13, wherein the amino acid is arginine and the metal ion is zinc.

15. A non-cross linked hyaluronic acid prepared by culturing Streptococcus dysgalactiae strain ID9103 (KCTC11818BP) in a medium comprising a carbon source and a nitrogen source for use in inhibiting adhesion.

16. The non-cross linked hyaluronic acid of claim 15, wherein the carbon source is maltose and the nitrogen source is casein enzymatic hydrolysate.

17. The non-cross linked hyaluronic acid of claim 15, wherein the medium further comprises an amino acid or a metal ion.

18. The non-cross linked hyaluronic acid of claim 17, wherein the amino acid is arginine and the metal ion is zinc.
